(19) United States
(12) Patent Application Publication
(10) Pub. No.: US 2011/0092941 A1
(43) Pub. Date: Apr. 21, 2011

(54) Alignment of Leg and Waist Bands on Disposable Absorbent Articles

(76) Inventors: Marcille Faye Ruman, Oshkosh, WI (US); David Fleger Bishop, Appleton, WI (US); Julia Hartono, Greenville, WI (US); Katherine Carol Wheeler, Menasha, WI (US); Wendy Lynn VanDyke, Appleton, WI (US); Terri Lynn Meisel, Appleton, WI (US); Robert Lee Popp, Hortonville, WI (US); Kathleen Irene Bennett, Neenah, WI (US)

(21) Appl. No.: 12/580,529

(22) Filed: Oct. 16, 2009

(51) Int. Cl. A61F 13/45; B32B 38/18

(52) U.S. Cl. 604/385.23; 156/64

(57) ABSTRACT

A method is provided for manufacturing a disposable absorbent article having multiple printed components, the method including positioning a first side panel adjacent a chassis such that first side panel medial edge is adjacent the chassis lateral edge, wherein the outer cover portion and the first side panel portion of a printed band feature each have a distal edge and a proximal edge, and wherein manufacturing is controlled such that a misalignment offset of either the distal edge of the outer cover portion and the distal edge of the first side panel portion, or the proximal edge of the outer cover portion and the proximal edge of the first side panel portion, of up to 100 percent of a longitudinal height of the outer cover portion is allowable and does not result in culling the article

### BACKGROUND

The present disclosure relates to aligning one or more printed components in a composite article, and to reducing manufacturing waste in producing the composite article. Strict alignment of article components, and the graphics and patterns printed thereon, has been the goal for manufacturers in an attempt to produce the most garment-like appearance in disposable absorbent articles.

A vast number of applications exist in which it is necessary or desirable to monitor the presence and/or position of one or more components of a composite article during manufacturing. For instance, in a largely automated process for manufacturing disposable absorbent articles such as diapers and other incontinence articles, it has long been the industry standard that certain components (e.g., support layers, absorbent pads, elastic components, fastener components, etc.) must be positioned or aligned with respect to each other and/or other components to produce an acceptable article. Prior art processes in this field are directed toward minimizing any misalignment between components themselves and between the printing thereon. Accordingly, inspection systems are commonly used to detect the positions of such components during manufacturing. If an inspection system determines that one or more components or their printing are out of position and thus do not properly register with other components and printing, the inspection system typically outputs one or more signals indicating that those articles should be culled and discarded.

### SUMMARY

Although situations exist in which such strict alignment is desirable, the processes necessary to accomplish such strict alignment also contribute to a great amount of wasted product as any article that does not exhibit such strict alignment is culled as unusable and discarded. The present disclosure provides for processes and articles that greatly reduce the amount of waste in the manufacture of disposable absorbent articles having multiple printed components.

Solutions to these problems are presented in a method for manufacturing a disposable absorbent article having multiple printed components, the method including producing a chassis having an outer cover including an outer cover portion of a first printed band feature, a liner generally parallel to the outer cover, and an absorbent core disposed therebetween, the chassis having a chassis first lateral edge, a chassis second lateral edge, and a chassis waist edge; and producing a first side panel including a first side panel portion of a first printed band feature, the first side panel having a first side panel waist edge and a first side panel medial edge. The method also includes positioning the first side panel adjacent the chassis such that first side panel medial edge is adjacent the chassis lateral edge, wherein the outer cover portion and the first side panel portion each have a distal edge and a proximal edge, and wherein the producing and positioning steps are controlled such that a misalignment offset of either the distal edge of the outer cover portion and the distal edge of the first side panel portion, or the proximal edge of the outer cover portion and the proximal edge of the first side panel portion, of up to 100 percent of a longitudinal height of the outer cover portion is allowable and does not result in culling the article.

In addition, a disposable absorbent article having multiple printed components includes a chassis having a front waist region, and rear waist region, an outer cover including a front waist region outer cover portion and rear waist region outer cover portion of a printed waistband, a liner generally parallel to the outer cover, and an absorbent core disposed therebetween, the chassis having a chassis first lateral edge, a chassis second lateral edge, and a chassis waist edge. The article also includes a first side panel including a first side panel portion of the printed waistband , the first side panel having a first side panel waist edge and a first side panel medial edge, the first side panel disposed adjacent the chassis such that first side panel medial edge is affixed to the chassis lateral edge, wherein the front waist region outer cover portion and the first side panel portion each have a distal edge and a proximal edge, and wherein either the distal edge of the outer cover portion and the distal edge of the first side panel portion have a misalignment offset of up to 100 percent of the average waistband height, or the proximal edge of the outer cover portion and the proximal edge of the first side panel portion have a misalignment offset of up to 100 percent of the average waistband height, where the average waistband height is the average of the longitudinal height of the front waist region outer cover portion and the longitudinal height of the rear waist region outer cover portion.

Further, a disposable absorbent article having multiple printed components includes a chassis having an outer cover including an outer cover portion of a first printed band feature, a liner generally parallel to the outer cover, and an absorbent core disposed therebetween, the chassis having a chassis first lateral edge, a chassis second lateral edge, and a chassis waist edge. The article also includes a first side panel including a first side panel portion of a first printed band feature, the first side panel having a first side panel waist edge and a first side panel medial edge, the first side panel disposed adjacent the chassis such that first side panel medial edge is affixed to the chassis lateral edge, wherein the outer cover portion and the first side panel portion each have a longitudinal height, and wherein the longitudinal height of the outer cover portion and the longitudinal height of the first side panel portion are different by at least 25 percent of the longitudinal height of the outer cover portion.

Additionally, a disposable absorbent article having multiple printed components includes a chassis having a front waist region, and rear waist region, an outer cover including a front waist region outer cover portion and rear waist region outer cover portion of a printed waistband, a liner generally parallel to the outer cover, and an absorbent core disposed therebetween, the chassis having a chassis first lateral edge, a chassis second lateral edge, and a chassis waist edge. The article also includes a first side panel including a first side panel portion of the printed waistband , the first side panel having a first side panel medial edge and a first side panel lateral edge, the first side panel disposed adjacent the chassis such that first side panel medial edge is affixed to the chassis lateral edge. The article also includes a second side panel including a second side panel portion of the printed waistband, the second side panel having a second side panel medial edge and a second side panel lateral edge, the second side panel disposed adjacent the chassis such that second side panel medial edge is affixed to the chassis lateral edge and the second side panel lateral edge is coupled to the first side panel lateral edge, wherein the first side panel portion and the second side panel portion each have a distal edge and a proximal edge, and wherein either the distal edge of the first side panel portion and the distal edge of the second side panel portion have a misalignment offset of up to 175 percent of the average waistband height, or the proximal edge of the first side panel portion and the proximal edge of the second side panel portion have a misalignment offset of up to 175 percent of the average waistband height, where the average waistband height is the average of the longitudinal height of the front waist region outer cover portion and the longitudinal height of the rear waist region outer cover portion

Other features of the disclosure will be in part apparent and in part pointed out hereinafter. Other objects and advantages of the present disclosure will become more apparent to those skilled in the art in view of the following description and the accompanying drawings.

### BRIEF DESCRIPTION OF THE DRAWINGS

The present disclosure will be more fully understood, and further features will become apparent, when reference is made to the following detailed description and the accompanying drawings. The drawings are merely representative and are not intended to limit the scope of the claims.

FIG. 1 is a side elevation of a child's pants with a fastening system of the pants shown connected on one side of the pants and disconnected on the other side of the pants;

FIG. 2 is a bottom plan view of the pants of FIG. 1 in an unfastened, stretched and laid flat condition to show the surface of the pants which faces away from the wearer;

FIG. 3 is a top plan view of the pants in its unfastened, stretched and laid flat condition to show the surface of the pants which faces the wearer when the pants are worn, with portions of the pants being cut away to reveal underlying features;

FIG. 4 is a schematic view of the pants of FIG. 1 showing a waistband and leg bands;

FIG. 5 is a schematic view of the pants of FIG. 1 showing a waistband and leg bands in various offset conditions;

FIG. 6 is a schematic view of the pants of FIG. 1 showing a waistband and leg bands in various offset conditions; and

FIG. 7 is a schematic view of the pants of FIG. 1 showing a waistband and leg bands in various offset conditions.

Repeat use of reference characters in the present specification and drawings is intended to represent the same or analogous features or elements of the present disclosure. The drawings are representational and are not necessarily drawn to scale. Certain proportions thereof might be exaggerated, while others might be minimized.

### DETAILED DESCRIPTION

It is to be understood by one of ordinary skill in the art that the present discussion is a description of exemplary aspects of the present disclosure only, and is not intended as limiting the broader aspects of the present disclosure.

In a largely automated process for manufacturing disposable absorbent articles such as diapers and other incontinence products, it has long been the industry standard that certain components (e.g., side panels, support layers, absorbent pads, elastic components, fastener components, etc.) must be positioned or aligned with respect to each other and/or other components to produce an acceptable article, particularly if those components are printed. Prior art processes in this field are directed toward minimizing any misalignment between components themselves and between the printing thereon. Accordingly, inspection systems are commonly used to detect the positions of such components during manufacturing. If an inspection system determines that one or more components or their printing are out of position and thus do not properly register with other components and printing, the inspection system typically outputs one or more signals indicating that those articles should be culled and discarded.

Although situations exist in which such complete alignment is desirable, the processes necessary to accomplish such complete alignment also contribute to a great amount of wasted product as any article that does not exhibit such strict alignment is culled as unusable and discarded. The present disclosure provides for processes and articles that greatly reduce the amount of waste in the manufacture of disposable absorbent articles having multiple printed components.

A number of discoveries provide a basis for the present disclosure. With respect to youth pants, there is the quite unexpected result that while complete alignment of components and their printing can be important to a parent's perception that a disposable absorbent article has an underwear-like appearance, such complete alignment, or a lack thereof, has little influence on the child's/wearer's perception that the article has an underwear-like appearance.

It was demonstrated in a study of waistband appearance that a parent's overall appearance rankings from most liked to least liked can be driven by the amount of offset apparent between the portion of the waistband that is disposed on a chassis versus the portion of the waistband that is disposed on a side panel. Such a waistband offset can also influence the rankings of the preference for the articles, along with the rankings of the perception of underwear-like appearance for most parents. Typically, parents rate any amount of waistband offset as being less desirable for appearance when compared to articles with no waistband offset.

Conversely, children/wearers are far less critical than the parents when rating an article's appearance from most to least liked. Children/wearers do not report articles with waistband offsets as being less underwear-like in their appearance than articles without a waistband offset. Conventional wisdom discounts giving weight to the perception of children/wearers because parents are the ones who purchase the articles.

Conventional wisdom, however, does not account for the fact that such articles are not purchased and used in the manner assumed by conventional wisdom. Typically moms purchase a package of such articles and bring the package home to the child/wearer. The child/wearer then removes the articles from the package and uses the articles in a relatively private manner. The percentage of parents who actually see the articles on their children for this age group is quite low. As a result, an opportunity exists for catering more to the perceptions of the child/wearer than to those of a parent.

Because a misalignment of printed components is largely transparent to a child/wearer, substantially greater misalignment can be allowed in what is defined as an acceptable article, thus leading to a substantial reduction in culled and discarded articles. Reducing the number of articles culled also substantially reduces the amount of manufacturing waste endemic with complete alignment requirements, and reduces the overall cost to the consumer

As an example, a significant source of variation in offsets occurs as a result of material splices where one roll of material ends and another is substituted. At one point in an article-manufacturing process, three different materials can be spliced into the article line—right side panel, left side panel, and an outercover. Because the article machine makes multiple bags of product per minute and splices can occur several times per hour, a tremendous amount of waste can be generated. Hypothetically, a machine running at 500 feet per minute makes 250 articles per minute. If lining out a splice takes 5 minutes (1250 articles), and splices for each of the three materials occur every 30 minutes (up to 6 in one hour), then at least 7500 of 15,000 articles per hour will be subject to being unacceptable and thus culled with a complete alignment requirement. The process could be producing unacceptably offset articles much of the time. Further opportunities for unacceptable offsets occur with potential variation in registration of the printed raw materials from roll to roll. As a result, moving away from a complete alignment requirement prevents a substantial amount of manufacturing waste.

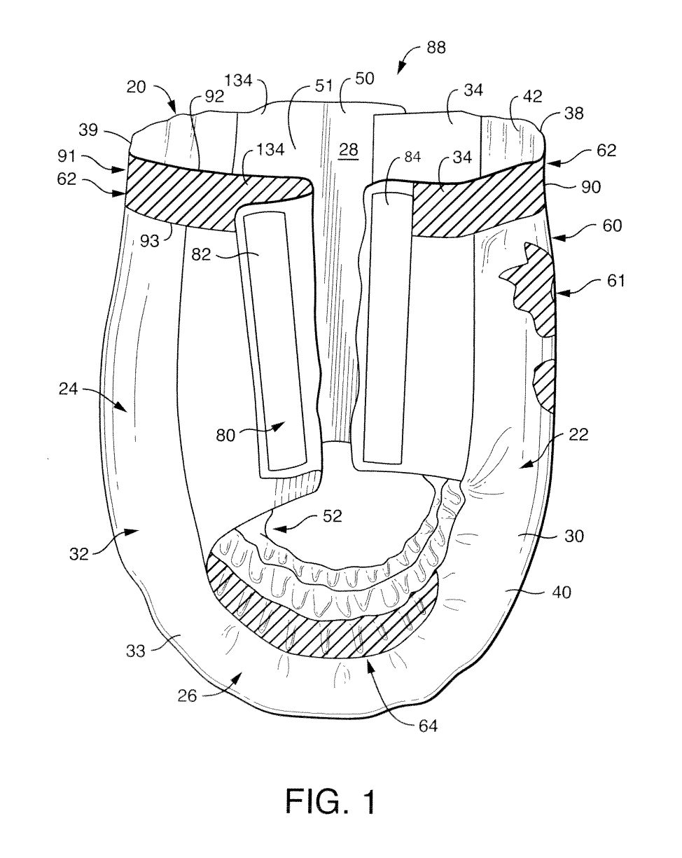

The methods and apparatus of the present disclosure can be used to make a variety of pre-fastened articles such as disposable absorbent garments including diapers, training pants, feminine hygiene products, incontinence products, medical garments, other personal care or health care garments, swim pants, athletic clothing, pants and shorts, and the like. More particularly, the methods and apparatus of the present disclosure can be used to make articles in which at least two elements of the article are connected together during the making thereof to assemble or “pre-fasten” the article. For ease of explanation, the methods and apparatus of the present disclosure are hereafter described in connection with making pre-fastened child's pants, generally indicated as 20 in FIG. 1. In particular, the methods and apparatus will be described in terms of those for making pre-fastened disposable pants as described in U.S. patent application Ser. No. 09/444,083 titled “Absorbent Articles With Refastenable Side Seams” and filed Nov. 22, 1999 (corresponding to PCT application WO 00/37009 published Jun. 29, 2000) by A. L. Fletcher et al., the disclosure of which is incorporated herein by reference. Pants 20 can also be constructed using the methods and apparatus disclosed in U.S. Pat. No. 4,940,464 issued Jul. 10, 1990 to Van Gompel et al.; and U.S. Pat. No. 5,766,389 issued Jun. 16, 1998 to Brandon et al.; the disclosures of which are also incorporated herein by reference.

It should be understood that as used herein, the term “component” includes not only discrete objects, but also objects yet to be formed into discrete objects (e.g., objects yet to be severed into discrete objects from a continuous sheet or web of material), particles (e.g., superabsorbent particles or polymers), adhesives, lotions, ointments, and other substances, as well as portions or characteristics of any such components including, for example, fold lines, bond lines (e.g., ultrasonic bond lines), bonded or adhered regions, and registration marks applied to or about components for subsequent detection during a manufacturing or inspection process.

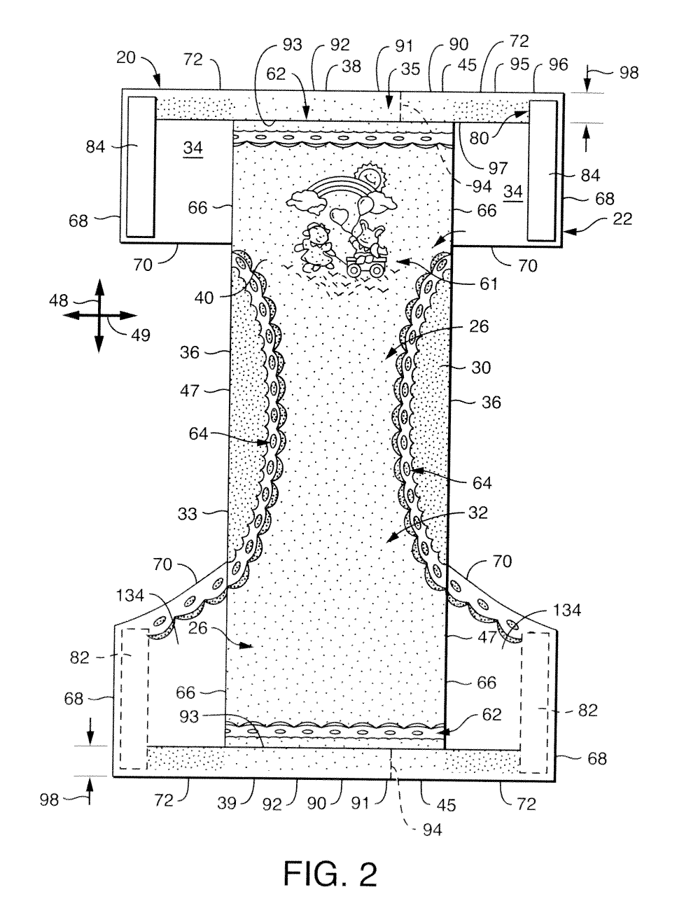

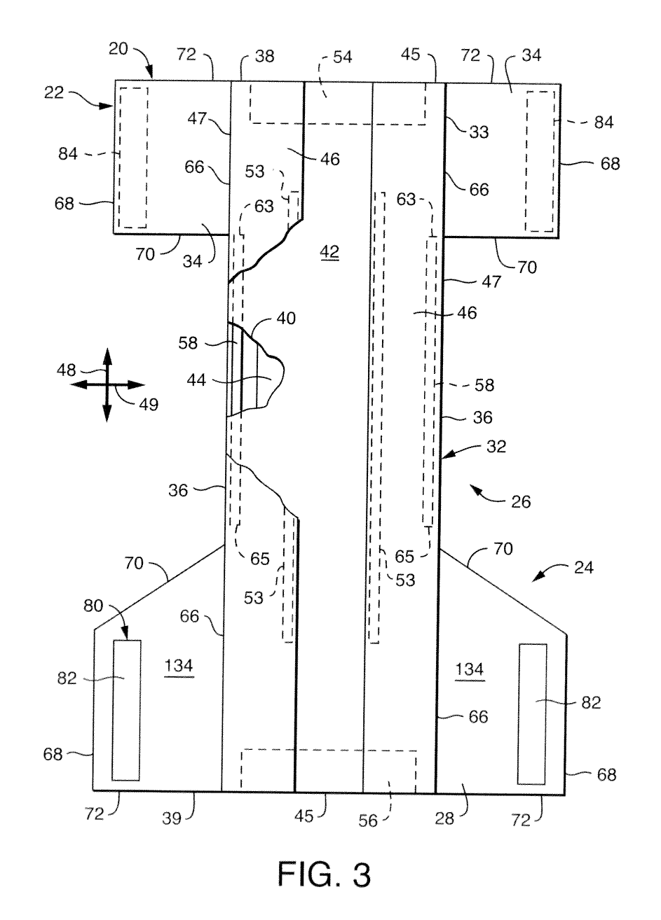

With reference now to the drawings, and in particular to FIG. 1, a pair of pants 20 is illustrated in a partially fastened condition and include an absorbent chassis 32 having a front waist region 22, a back waist region 24, a crotch region 26 interconnecting the front and back waist regions, an inner surface 28 which is configured to contact the wearer, and an outer surface 30 opposite the inner surface and configured to contact the wearer's clothing. With additional reference to FIGS. 2 and 3, the absorbent chassis 32 also has a pair of laterally opposite side edges 36 and a pair of longitudinally opposite waist edges, respectively designated front waist edge 38 and back waist edge 39. The front waist region 22 is contiguous with the front waist edge 38, and the back waist region 24 is contiguous with the back waist edge 39.

The illustrated absorbent chassis 32 includes a composite structure 33 (FIGS. 2 and 3), which when laid flat can be rectangular or any other desired shape, and has a pair of laterally opposite front side panels 34 and a pair of laterally opposite back side panels 134 extending outward therefrom. Each side panel 34, 134 includes a side panel central region 76 positioned generally on each side panel 34, 134, and does not include a waistband or leg bands (FIG. 2). The side panel central region 76 has a side panel central region appearance, which is the physical appearance of the side panel central region 76.

The composite structure 33 and side panels 34, 134 can include two or more separate elements, as shown in FIG. 1, or be integrally formed. Integrally formed side panels 34, 134 and composite structure 33 would include at least some common materials, such as the bodyside liner, flap composite, outer cover, other materials and/or combinations thereof, and could define a one-piece elastic, stretchable, or non-stretchable pants. The illustrated composite structure 33 includes an outer cover 40, a bodyside liner 42 (FIGS. 1 and 3) connected to the outer cover in a superposed relation, an absorbent assembly 44 (FIG. 3) disposed between the outer cover and the bodyside liner, and a pair of containment flaps 46 (FIG. 3). The illustrated composite structure 33 has opposite ends 45 (FIGS. 2 and 3) that form portions of the front and back waist edges 38 and 39, and opposite side edges 47 which form portions of the side edges 36 of the absorbent chassis 32 (FIGS. 2 and 3). The outer cover 42 includes an outer cover central region 75 positioned generally in the front and/or back waist regions 22, 24, and does not include a waistband or leg bands (FIG. 2). The outer cover central region 75 has an outer cover central region appearance, which is the physical appearance of the outer cover central region 75.

For reference, arrows 48 and 49 (FIGS. 2 and 3) depict the orientation of the longitudinal axis and the transverse or lateral axis, respectively, of the pants 20.

With the pants 20 in the fastened position as partially illustrated in FIG. 1, the front and back side panels 34, 134 are connected together by a fastening system 80 to define a three-dimensional pants configuration having an interior space 51, a waist opening 50 for receiving the wearer into the interior space of the pants, a pair of leg openings 52 and engagement seams 88 along which the side panels 34, 134 are connected. The interior space 51 of the pants 20 is thus bounded by the absorbent chassis 32, the engagement seams 88 and the portions of the side panels 34, 134 extending on opposite sides of the engagement seams 88 (e.g., between the engagement seams 88 and the absorbent chassis 32). As used herein, the “interior space” 51 is intended to refer to the space between any two portions of a three-dimensional article which generally oppose each other. It is understood that a transverse cross-section of the article need not be closed, e.g., continuous, to define the interior space 51. For example, a two-dimensional article can be generally folded over on itself so that two portions of the article oppose each other to define an interior space of the article therebetween. Thus, the interior space 51 of the pants 20 shown in FIG. 1 can be defined by the side panels 34, 134 themselves or, if the side panels are fully straightened therebetween, the interior space is defined by a combination of the side panels and the front and back waist regions 22, 24 of the absorbent chassis 32.

The front waist region 22 includes the portion of the pants 20 which, when worn, is positioned on the front of the wearer while the back waist region 24 includes the portion of the pants which, when worn, is positioned on the back of the wearer. The crotch region 26 of the pants 20 includes the portion of the pants 20 which, when worn, is positioned between the legs of the wearer and covers the lower torso of the wearer. The front and back side panels 34 and 134 include the portions of the pants 20 which, when worn, are positioned on the hips of the wearer. The waist edges 38 and 39 of the absorbent chassis 32 are configured to encircle the waist of the wearer when worn and together define the waist opening 50 (FIG. 1). Portions of the side edges 36 in the crotch region 26 generally define the leg openings 52.

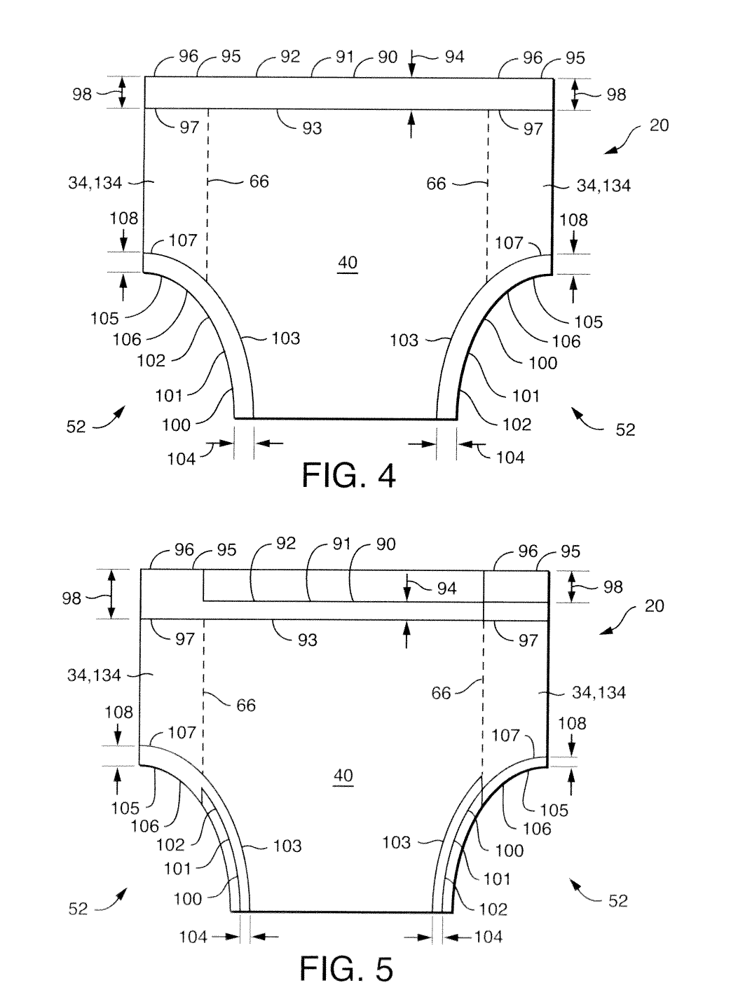

In another aspect of the present disclosure best illustrated in FIGS. 2 and 4, the pants 20 also include a printed waistband 90. The printed waistband 90 includes a waistband outer cover portion 91 disposed on the absorbent chassis 32 adjacent the waist opening 50. The waistband outer cover portion 91 includes a distal edge 92, a proximal edge 93, and a longitudinal height 94, which is the longitudinal distance between the proximal edge 93 and the distal edge 92. The printed waistband 90 also includes a waistband first side panel portion 95 disposed on one of the side panels 34, 134 adjacent the waist opening 50. The waistband first side panel portion 95 includes a distal edge 96, a proximal edge 97, and a longitudinal height 98, which is the longitudinal distance between the proximal edge 97 and the distal edge 96. In addition, each other article component adjacent the waist opening 50 can have a portion of the printed waistband 90 with similar features.

In practice, the distal edges 92, 96 of the waistband outer cover portion 91 and of the waistband first side panel portion 95 can be longitudinally aligned or can be longitudinally offset. The proximal edges 93, 97 of the waistband outer cover portion 91 and of the waistband first side panel portion 95 can be longitudinally aligned or can be longitudinally offset. Offsets, misalignment, and misalignment offsets refer to the longitudinal positioning of an edge of a component or a feature as compare to the longitudinal positioning of an edge of another component or feature.

In addition, the longitudinal heights 94, 98 of the waistband outer cover portion 91 and of the waistband first side panel portion 95 can be equal or unequal. Further, the waistband outer cover portion 91 and the waistband first side panel portion 95 can have no lateral gap therebetween in the lateral direction, or can be disposed such that a lateral gap is present between the portions.

In a specific example, the proximal edge 93 of a waistband outer cover portion 91 can be 25 mm from the waist edge 38, 39 of the pants 20, while the distal edge 92 of the waistband outer cover portion 91 can be 0 mm from the waist edge 38, 39 of the pants 20. Similarly, the proximal edge 97 of the waistband first side panel portion 95 can be 15 mm from the waist edge 38, 39 of the pants 20, and the distal edge 96 of the waistband first side panel portion 95 can be 5 mm from the waist edge 38, 39 of the pants 20. In this example, proximal edge 93 of the waistband outer cover portion 91 is offset from the proximal edge 97 of the waistband first side panel portion 95 by 10 mm. The distal edge 92 of the waistband outer cover portion 91 is offset from the distal edge 96 of the waistband first side panel portion 95 by 5 mm. Further in this example, the longitudinal height 94 of the waistband outer cover portion 91 is 25 mm, and the longitudinal height 98 of the waistband first side panel portion 95 is 10 mm. This produces a longitudinal height difference between the two portions of 15 mm.

In another aspect of the present disclosure best illustrated in FIG. 4, the pants 20 also include a printed leg band 100. The printed leg band 100 includes a leg band outer cover portion 101 disposed on the absorbent chassis 32 adjacent the leg opening 52. The leg band outer cover portion 101 includes a distal edge 102, a proximal edge 103, and a longitudinal height 104, which is the longitudinal distance between the proximal edge 103 and the distal edge 102. The printed leg band 100 also includes a leg band first side panel portion 105 disposed on one of the side panels 34, 134 adjacent the leg opening 52. The leg band first side panel portion 105 includes a distal edge 106, a proximal edge 107, and a longitudinal height 108, which is the longitudinal distance between the proximal edge 107 and the distal edge 106. In addition, each other article component adjacent the leg opening 52 can have a portion of the printed leg band 100 with similar features. The pants 20 preferably includes a printed leg band 100 encircling each leg opening 52.

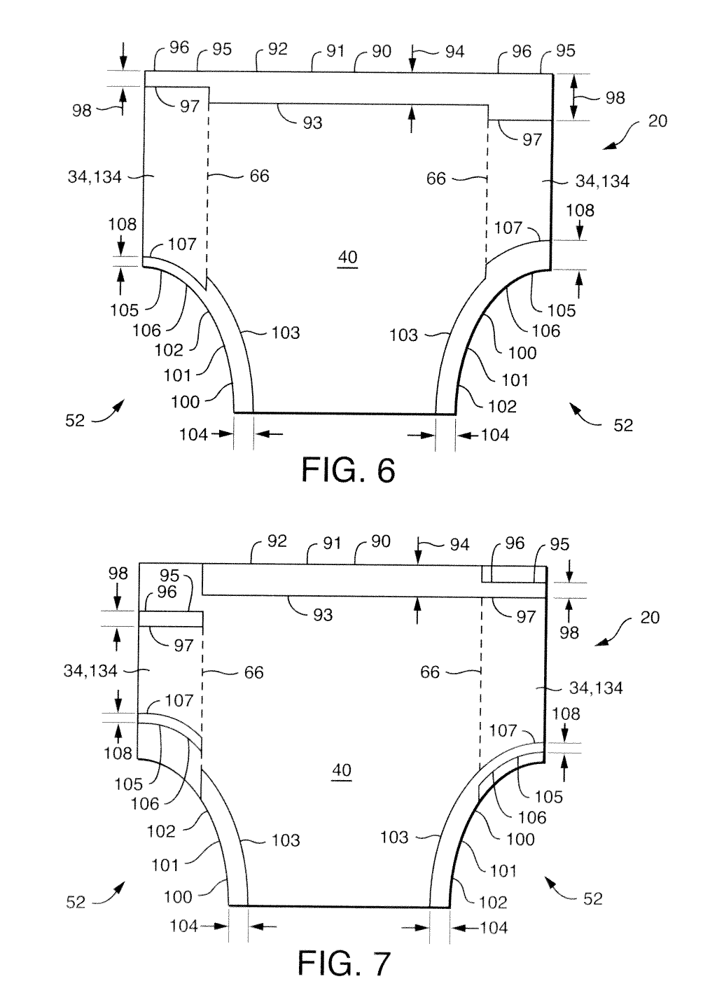

Various configurations of alignments, misalignment, and differences in distal edges, proximal edges, and longitudinal heights are illustrated in FIGS. 4-7.

As used herein, the longitudinal height of a printed waistband 90 or of a printed leg band 100 is generally the distance between the distal and proximal edges of that band. Some bands, however, do not have linear edges. For example, bands can be scalloped, sinusoidal, arcuate, or of any other non-linear design. In such cases, the longitudinal height is the greatest distance between a distal edge and a proximal edge.

In practice, the distal edges 102, 106 of the leg band outer cover portion 101 and of the leg band first side panel portion 105 can be longitudinally aligned or can be longitudinally offset. The proximal edges 103, 107 of the leg band outer cover portion 101 and of the leg band first side panel portion 105 can be longitudinally aligned or can be longitudinally offset. In addition, the longitudinal heights 104, 108 of the leg band outer cover portion 101 and of the leg band first side panel portion 105 can be equal or unequal. Further, the leg band outer cover portion 101 and the leg band first side panel portion 105 can have no lateral gap therebetween in the lateral direction, or can be disposed such that a lateral gap is present between the portions.

The absorbent chassis 32 is configured to contain and/or absorb any exudates discharged from the wearer. For example, the absorbent chassis 32 desirably although not necessarily includes the pair of containment flaps 46 which are configured to provide a barrier to the transverse flow of body exudates. A flap elastic member 53 (FIG. 3) can be operatively joined with each containment flap 46 in any suitable manner as is well known in the art. The elasticized containment flaps 46 define an unattached edge which assumes an upright configuration in at least the crotch region 26 of the pants 20 to form a seal against the wearer's body. The containment flaps 46 can be located along the side edges 36 of the absorbent chassis 32, and can extend longitudinally along the entire length of the absorbent chassis 32, or can only extend partially along the length of the absorbent chassis 32. Suitable constructions and arrangements for the containment flaps 46 are generally well known to those skilled in the art and are described in U.S. Pat. No. 4,704,116 issued Nov. 3, 1987 to Enloe, which is incorporated herein by reference.

To further enhance containment and/or absorption of body exudates, the pants 20 desirably although not necessarily include a front waist elastic member 54, a rear waist elastic member 56, and leg elastic members 58, as are known to those skilled in the art (FIG. 3). The waist elastic members 54 and 56 can be operatively joined to the outer cover 40 and/or the bodyside liner 42 along the opposite waist edges 38 and 39, and can extend over part or all of the waist edges. The leg elastic members 58 can be operatively joined to the outer cover 40 and/or the bodyside liner 42 along the opposite side edges 36 and positioned in the crotch region 26 of the pants 20. The leg elastic members 58 can be longitudinally aligned along each side edge 47 of the composite structure 33. Each leg elastic member 58 has a front terminal point 63 and a back terminal point 65, which represent the longitudinal ends of the elastic gathering caused by the leg elastic members. The front terminal points 63 can be located adjacent the longitudinally innermost parts of the front side panels 34, and the back terminal points 65 can be located adjacent the longitudinally innermost parts of the back side panels 134.

As shown in FIGS. 1 and 2, the pants 20 and in particular the outer cover 40 desirably includes one or more appearance-related components. Examples of appearance-related components include, but are not limited to, graphics; highlighting or emphasizing leg and waist openings in order to make product shaping more evident or visible to the user; highlighting or emphasizing areas of the product to simulate functional components such as elastic leg bands, elastic waistbands, simulated “fly openings” for boys, ruffles for girls; highlighting areas of the product to change the appearance of the size of the product; registering wetness indicators, temperature indicators, and the like in the product; registering a back label, or a front label, in the product; and registering written instructions at a desired location in the product.

The illustrated pair of pants 20 is designed for use by young girls and includes a registered outer cover graphic 60 (FIGS. 1 and 2). In this design, the registered graphic 60 includes a primary pictorial image 61, simulated waist ruffles 62, and simulated leg ruffles 64. The primary pictorial image 61 includes an object graphic such as a rainbow, sun, clouds, animal characters, wagon and balloons. Any suitable design can be utilized for pants intended for use by young girls, so as to be aesthetically and/or functionally pleasing to them and the caregiver. The appearance-related components are desirably positioned on the pants 20 at selected locations, which can be carried out using the methods disclosed in U.S. Pat. No. 5,766,389 issued Jun. 16, 1998 to Brandon et al., the entire disclosure of which is incorporated herein by reference. The primary pictorial image 61 is desirably positioned in the front waist region 22 along the longitudinal center line of the pants 20.

The printed graphics on the pants 20 can share a color palette, can be of the same color, can be of complementary colors, or can follow any suitable color scheme. Different areas of the pants 20 can also be printed with similar or identical graphical elements such as stars, circles, butterflies, etc. Different areas of the pants 20 can also be printed with similar or identical graphics that share a graphic theme. For example, the pants 20 can be printed with flowers that might or might not be identical in size, type, color, etc., but that all share the flower theme. In another aspect, the article can be reverse printed such that graphical elements, for example, appear as an absence of printing.

As noted previously, the illustrated pants 20 have front and back side panels 34 and 134 disposed on each side of the absorbent chassis 32. The front side panels 34 can be permanently bonded along seams 66 to the composite structure 33 of the absorbent chassis 32 in the respective front and back waist regions 22 and 24. More particularly, as seen best in FIGS. 2 and 3, the front side panels 34 can be permanently bonded to and extend transversely outward beyond the side edges 47 of the composite structure 33 in the front waist region 22, and the back side panels 134 can be permanently bonded to and extend transversely outward beyond the side edges of the composite structure in the back waist region 24. The side panels 34 and 134 can be bonded to the composite structure 33 using attachment means known to those skilled in the art such as adhesive, thermal or ultrasonic bonding. Alternatively, the side panels 34 and 134 can be formed as an integral portion of a component of the composite structure 33. For example, the side panels can include a generally wider portion of the outer cover 40, the bodyside liner 42, and/or another component of the absorbent chassis 32. The front and back side panels 34 and 134 can be permanently bonded together or be releasably connected with one another such as by the fastening system 80 of the illustrated aspect.

As best illustrated in FIGS. 2 and 3, the front and back side panels 34, 134 each have an outer edge 68 spaced laterally from the seam 66, a leg end edge 70 disposed toward the longitudinal center of the pants 20, and a waist end edge 72 disposed toward a longitudinal end of the pants. The leg end edge 70 and waist end edge 72 extend from the side edges 47 of the composite structure 33 to the outer edges 68. The leg end edges 70 of the side panels 34 and 134 form part of the side edges 36 of the absorbent chassis 32. In the back waist region 24, the leg end edges 70 are desirably although not necessarily curved and/or angled relative to the transverse axis 49 to provide greater coverage toward the back of the pants 20 as compared to the front of the pants. The waist end edges 72 are desirably parallel to the transverse axis 49. The waist end edges 72 of the front side panels 34 form part of the front waist edge 38 of the absorbent chassis 32, and the waist end edges 72 of the back side panels 134 form part of the back waist edge 39 of the absorbent chassis. The waist end edges 72 are generally aligned or co-linear with the front and back waist edges 38, 39.

In particular aspects for improved fit and appearance, the side panels 34, 134 desirably have an average length measured parallel to the longitudinal axis 48 which is about 15 percent or greater, and particularly about 25 percent or greater, of the overall length of the pants, also measured parallel to the longitudinal axis 48. For example, in pants 20 having an overall length of about 54 centimeters, the side panels 34, 134 desirably have an average length of about 10 centimeters or greater, such as about 15 centimeters. While each of the side panels 34, 134 extends from the waist opening 50 to one of the leg openings 52, the illustrated back side panels 134 have a continually decreasing length dimension moving from the seam 66 to the outer edge 68, as is best shown in FIGS. 2 and 3.

Each of the side panels 34, 134 can include one or more individual, distinct pieces of material. In particular aspects, for example, each side panel 34, 134 can include first and second side panel portions that are joined at a seam, or can include a single piece of material which is folded over upon itself (not shown).

The side panels 34, 134 desirably although not necessarily include an elastic material capable of stretching in a direction generally parallel to the transverse axis 49 of the pants 20. Suitable elastic materials, as well as one process of incorporating elastic side panels into pants, are described in the following U.S. Pat. No.: 4,940,464 issued Jul. 10, 1990 to Van Gompel et al.; U.S. Pat. No. 5,224,405 issued Jul. 6, 1993 to Pohjola; U.S. Pat. No. 5,104,116 issued Apr. 14, 1992 to Pohjola; and U.S. Pat. No. 5,046,272 issued Sep. 10, 1991 to Vogt et al.; all of which are incorporated herein by reference. An alternative elastic material is described below. In particular aspects, the elastic material includes a stretch-thermal laminate (STL), a neck-bonded laminate (NBL), a reversibly necked laminate, or a stretch-bonded laminate (SBL) material. Methods of making such materials are well known to those skilled in the art and described in U.S. Pat. No. 4,663,220 issued May 5, 1987 to Wisneski et al.; U.S. Pat. No. 5,226,992 issued Jul. 13, 1993 to Morman; and European Patent Application No. EP 0 217 032 published on Apr. 8, 1987 in the names of Taylor et al.; all of which are incorporated herein by reference. Alternatively, the side panel material can include other woven or nonwoven materials, such as those described above as being suitable for the outer cover 40 or bodyside liner 42; mechanically pre-strained composites; or stretchable but inelastic materials.

Further detail with respect to elastic laminates of the present disclosure can be found in co-pending U.S. Patent Publication No. 2008/0095978 entitled “Nonwoven Composite Containing An Apertured Elastic Film,” which is incorporated herein by reference to the extent it does not conflict herewith.

Pants 20 can have the side panels 34, 134 affixed to each other in for securing the pants 20 about the waist of the wearer. The side panels 34, 134 can be affixed by bonding, mechanical fasteners, or any other suitable method, and can be affixed permanently, in a tearable manner, or in a refastenable manner. The illustrated pants 20 includes the fastening system 80 for refastenably securing the pants about the waist of the wearer. The illustrated fastening system 80 includes first fastening components 82 adapted for refastenable engagement to corresponding second fastening components 84. In one aspect, one surface of each of the first fastening components 82, 84 includes a plurality of engaging elements which project from that surface. The engaging elements of the first fastening components 82 are adapted to repeatedly engage and disengage engaging elements of the second fastening components 84.

The fastening components 82, 84 can comprise separate elements bonded to the side panels 134, 34, or they may be integrally formed with the side panels. Thus, unless otherwise specified, the term “fastening component” includes separate components which function as fasteners, and regions of materials such as the side panels 34, 134 which function as fasteners. Moreover, a single material can define multiple fastening components to the extent that different regions of the material function as separate fasteners. The fastening components 82, 84 can be located on the side panels 134, 34, between the side panels such as on the absorbent chassis, or a combination of the two.

Components, alignment, and articles can be inspected using an infrared inspection system such as that described in U.S. Pat. No. 6,885,451 to Vogt, et al., and in co-pending U.S. patent application Ser. No. ______, entitled “Disposable Absorbent Articles Incorporating Black-Free Colorant” each of which is incorporated herein by reference to the extent it does not conflict herewith.

As described above, waist and leg openings of disposable absorbent articles are often created by joining multiple components. The present disclosure applies in particular to articles for which the circumference of either the waist or leg opening has more than two interfaces or junctions at which components are attached, bonded, etc., as is described in more detail above. The article can include one or more printed band features in the form of a waistband partially or completely encircling the waist opening and/or a leg band partially or completely encircling one or each leg opening. For example, a fully encircling waistband is achieved by attaching a pair of right and left side panels to each of the front and rear waist regions of an article's outercover and then bonding the front and back assemblies together at the sides to create a closed garment. In this example, the waistband encompasses six components (with the outer cover counting twice with its front and rear waist regions) and six junctions. Aligning each of these components properly is a complex process that is further complicated if printing on some or all components also needs to be aligned. In other aspects, a functional waistband that does or does not span the entire waist circumference can be attached to the outercover and/or side panels with similar complexity. The printed band feature can be of uniform or any other suitable appearance. Multiple printed band features, such as a waistband including a plurality of generally horizontal and adjoining lines, are considered to be a single band feature

Similarly, a fully encircling leg band is also achieved by attaching a pair of right and left side panels to each of the front and rear waist regions of an article's outercover and then bonding the front and back assemblies together at the sides to create a closed garment. In this example, each leg band encompasses three components and three junctions. Aligning each of these components properly is a complex process that is further complicated if printing on some or all components also needs to be aligned. In other aspects, functional leg bands and leg elastics that do not span the entire leg circumference can be attached to the outercover and/or side panels.

Printing fully-encircling waist and leg bands provides a visual cue to a consumer of a fully-encircling functional waistband, which is a key component in giving the article an appearance of conventional underwear. Printing is leveraged to create the visual perception of a continuous waist and/or leg band material that exists across multiple pieces (e.g., side panels, outercover, waist elastics, and leg elastics). The printing can be applied to the garment or outward facing surfaces of the outer cover and side panels, the body or inward facing surfaces of the outercover and side panels, and/or sandwiched within material layers.

In an alternative aspect of the present disclosure, a waistband and/or a leg band is not required to have a distinct or abrupt edge. A gradation in the printing, such as a gradation from darker to lighter, can be employed for visual effect and, in some cases, to minimize the appearance of a misalignment offset. The same acceptable/unacceptable offset limits and features described herein can be applied to such a gradation with respect to its edges and/or longitudinal height.

The components can be printed prior to, during, or after the multiple components are combined to form the absorbent article. The printed waist/leg bands are substantially but not necessarily completely aligned at the seams (e.g., outercover to side panel, front to back side seams) and substantially encircle the waist and leg openings. The article can have additional printing or pigmentation to further enhance a more uniform, garment-like look.

In particular embodiments, after the first side panel is joined to the chassis to impart a pant-like configuration to the pants 20, the outer cover portion is in complete alignment with the first side panel portion. As used herein, “complete alignment” and variations thereof mean that the longitudinal height of the outer cover portion is the same as the longitudinal height of the first side panel portion, and further that the outer cover portion and the first side panel portion have no longitudinal offset from each other in the assembled pant-like configuration.

In other embodiments, after the first side panel is joined to the chassis to impart a pant-like configuration to the pants 20, the outer cover portion is in substantial alignment with the first side panel portion. As used herein, “substantial alignment” and variations thereof mean either that the longitudinal height of the outer cover portion is not the same as the longitudinal height of the first side panel portion, or that the outer cover portion and the first side panel portion are longitudinally offset from each other in the assembled pant-like configuration by the amounts described below.

To maintain an overall continuous look of the waist and leg band graphics, the components themselves should be aligned, and the printing on those components can be substantially but not necessarily completely aligned. During the article manufacturing process, materials are joined and seamed by any suitable method including ultrasonics, pressure, or adhesive bonding to form a continuous functional waistband or leg band. The printing on these components should have minimal offset, but complete alignment need not be sought. For example, the distal edges of the waistband outer cover portion and of the waistband first side panel portion should be longitudinally offset by less than 100% of longitudinal height of the waistband outer cover portion, preferably by less than 75% of that height, more preferably by less than 50% of that height, and most preferably by less than 25% of that height. For any given situation, a suitable acceptable offset limit can be set. In a more specific example, for a waistband longitudinal height of 25 mm, the offset should be less than 25 mm, preferably less than 18.75 mm, more preferably less than 12.5 mm, and most preferably less than 6.25 mm. Similar examples apply to the proximal edges of the waistband and the leg bands, to the distal edges of the waistband and the leg bands, and to the longitudinal heights of the waistband and the leg bands.

Another aspect of the present disclosure recognizes the insensitivity of the primary user of the articles to misalignment or offset as described above. In this aspect, an offset is set at which the article is still acceptable and thus not subject to culling and being discarded. For example, the distal edges of the waistband outer cover portion and of the waistband first side panel portion can be longitudinally offset as much as 25% of longitudinal height of the waistband outer cover portion, preferably by as much as 50% of that height, more preferably by as much as 75% of that height, and most preferably by as much as 100% of that height. For any given situation, a suitable acceptable offset limit can be set. In a more specific example, for a waistband longitudinal height of 25 mm, the offset can be as much as 6.25 mm, preferably as much as 12.5 mm, more preferably as much as 18.75 mm, and most preferably as much as 25 mm. Similar examples apply to the proximal edges of the waistband and the leg bands, to the distal edges of the waistband and the leg bands, and to the longitudinal heights of the waistband and the leg bands.

The outer cover portion and the first side panel portion can also be offset by differing in longitudinal height. Where the outer cover portion and the first side panel portion each have a longitudinal height, the longitudinal height of the outer cover portion and the longitudinal height of the first side panel portion can be different by at least 100 percent of the longitudinal height of the outer cover portion, preferably by at least 75 percent of that height, more preferably by at least 50 percent of that height, and most preferably by at least 25 percent of that height. For any given situation, a suitable acceptable offset limit can be set.

While an example of a waistband having a longitudinal height of 25 mm is described, waistbands and leg bands usually vary from 5 mm in 60 mm in longitudinal height depending on underwear style, size, intended user age, and gender emulated by the disposable articles.

Alternatively, limits similar to those described above can be set with respect to the average waistband height. The waistband can include a front waist region outer cover portion and a rear waist region outer cover portion each having a longitudinal height. The average waistband height is the average of the longitudinal height of the front waist region outer cover portion and the longitudinal height of the rear waist region outer cover portion. In one aspect, for example, the front waist region outer cover portion and the first side panel portion each have a distal edge, and the distal edge of the front waist region outer cover portion and the distal edge of the first side panel portion have a misalignment offset of as much as 25% of the average waistband height, preferably by as much as 50% of that height, more preferably by as much as 75% of that height, and most preferably by as much as 100% of that height. In another aspect, for example, the front waist region outer cover portion and the first side panel portion each have a proximal edge, and the proximal edge of the front waist region outer cover portion and the proximal edge of the first side panel portion have a misalignment offset of as much as 25% of the average waistband height, preferably by as much as 50% of that height, more preferably by as much as 75% of that height, and most preferably by as much as 100% of that height. For any given situation, a suitable acceptable offset limit can be set.

In another aspect of the present disclosure, the front waist region outer cover portion and the rear waist region outer cover portion should be of a minimum longitudinal height. For example, the longitudinal height of either the front waist region outer cover portion or the rear waist region outer cover portion should be at least 5 percent of the total waistband height, where the total waistband height is the sum of the longitudinal height of the front waist region outer cover portion and the longitudinal height of the rear waist region outer cover portion. In other aspects, the longitudinal height of either the front waist region outer cover portion or the rear waist region outer cover portion should be at least 10 percent of the total waistband height, at least 25 percent of the total waistband height, or at least 50 percent of the total waistband height. For any given situation, a suitable acceptable longitudinal height can be set.

In addition, as described above, the waistband and leg band portions can have no lateral gap therebetween in the lateral direction, or can be disposed such that a lateral gap is present between the portions. In a specific example, the lateral gap between the waistband outer cover portion and the waistband first side panel portion should be less than 15 mm, preferably less than 10 mm, more preferably less than 5 mm, and most preferably 0 mm. It should be noted that some printed waist band and leg bands, such as those with vertical stripes, can include an inherent lateral gap as part of their patterns. Such inherent lateral gaps are not included herein in the definition of a lateral gap.

It should be noted that all waistband and leg band offset and height limits, conditions, and features apply equally to the outer cover with respect to either of its adjoining side panels, or to any side panel with respect to its adjoining side panel or outer cover. The same limits and allowances in misalignment offset, longitudinal height, and longitudinal height differences can be applied in any of those situations to determine what is allowable and does not result in culling the article. Further, greater misalignment offset for the seam between side panels can be allowed because that seam is less visible. For example, the distal edges of the waistband first side panel portion and of the waistband second side panel portion can be longitudinally offset as much as 25% of longitudinal height of the waistband outer cover portion, preferably by as much as 75% of that height, more preferably by as much as 125% of that height, and most preferably by as much as 175% of that height. Similarly, the proximal edges of the waistband first side panel portion and of the waistband second side panel portion can be longitudinally offset as much as 25% of longitudinal height of the waistband outer cover portion, preferably by as much as 75% of that height, more preferably by as much as 125% of that height, and most preferably by as much as 175% of that height. For any given situation, a suitable acceptable offset limit can be set.

Additionally, an article constructed from multiple components can have a fully-encircling printed waistband printed on both the inside and outside of the article to mimic a functional waistband. The article can also have additional functional waistband materials and components that overlap the side panels. In this latter case, a printed waistband can also be added, and can be partially or completely printed on all or a portion of the functional waistband.

While a side panel and outer cover chassis design is disclosed above, the present disclosure can apply equally to conventional diapers, boxer-type disposable articles, pull-on underwear, adjustable underwear, disposable underwear, and belted shields.

When introducing elements of the present disclosure or the preferred aspect(s) thereof, the articles “a”, “an”, “the”, and “the” are intended to mean that there are one or more of the elements. The terms “comprising,” “including”, and “having” are intended to be inclusive and mean that there can be additional elements other than the listed elements.

The disclosure has been described with reference to various specific and illustrative aspects and techniques. However, it should be understood that many variations and modifications can be made while remaining within the spirit and scope of the disclosure. Many alternatives, modifications and variations will be apparent to those skilled in the art in light of the foregoing description. Accordingly, this disclosure is intended to embrace all such alternatives, modifications, and variations that fall within the spirit and scope of the appended claims.

1. A method for manufacturing a disposable absorbent article having multiple printed components, the method comprising:
producing a chassis having an outer cover including an outer cover portion of a first printed band feature, a liner generally parallel to the outer cover, and an absorbent core disposed therebetween, the chassis having a chassis first lateral edge, a chassis second lateral edge, and a chassis waist edge;
producing a first side panel including a first side panel portion of a first printed band feature, the first side panel having a first side panel waist edge and a first side panel medial edge; and
positioning the first side panel adjacent the chassis such that first side panel medial edge is adjacent the chassis lateral edge,
wherein the outer cover portion and the first side panel portion each have a distal edge and a proximal edge, and wherein the producing and positioning steps are controlled such that a misalignment offset of either the distal edge of the outer cover portion and the distal edge of the first side panel portion, or the proximal edge of the outer cover portion and the proximal edge of the first side panel portion, of up to 100 percent of a longitudinal height of the outer cover portion is allowable and does not result in culling the article.

2. The method of claim 1, wherein the printed band feature is a waistband.

3. The method of claim 1, wherein the printed band feature is a leg band.

4. The method of claim 1, wherein the chassis further includes a chassis portion of a second printed band feature, wherein the side panel further includes a first side panel portion of a second printed band feature, and wherein the first printed band feature is a waistband and the second printed band feature is a leg band.

5. The method of claim 1, further comprising:
producing a second side panel including a second side panel portion of a first printed band feature, the second side panel having a second side panel waist edge and a second side panel medial edge; and
positioning the second side panel adjacent the chassis such that second side panel medial edge is adjacent the chassis second lateral edge, and the second panel waist edge is generally co-linear with the chassis waist edge,
wherein the second side panel portion has a distal edge and a proximal edge, and wherein the producing and positioning steps are controlled such that a misalignment offset of either the distal edge of the outer cover portion and the distal edge of the second side panel portion, or the proximal edge of the outer cover portion and the proximal edge of the second side panel portion, of up to 100 percent of a longitudinal height of the outer cover portion is allowable and does not result in culling the article.

6. The method of claim 1, wherein a misalignment offset of either the distal edge of the outer cover portion and the distal edge of the first side panel portion, or the proximal edge of the outer cover portion and the proximal edge of the first side panel portion, of between 5 percent and 100 percent of a longitudinal height of the outer cover portion is allowable.

7. The method of claim 1, wherein a misalignment offset of either the distal edge of the outer cover portion and the distal edge of the first side panel portion, or the proximal edge of the outer cover portion and the proximal edge of the first side panel portion, of between 10 percent and 100 percent of a longitudinal height of the outer cover portion is allowable.

8. The method of claim 1, wherein a misalignment offset of either the distal edge of the outer cover portion and the distal edge of the first side panel portion, or the proximal edge of the outer cover portion and the proximal edge of the first side panel portion, of between 5 percent and 75 percent of a longitudinal height of the outer cover portion is allowable.

9. The method of claim 1, wherein a misalignment offset of either the distal edge of the outer cover portion and the distal edge of the first side panel portion, or the proximal edge of the outer cover portion and the proximal edge of the first side panel portion, of between 5 percent and 50 percent of a longitudinal height of the outer cover portion is allowable.

10. The method of claim 1, wherein the first panel waist edge is generally co-linear with the chassis waist edge.

11. A disposable absorbent article having multiple printed components, the article comprising:
a chassis having a front waist region, and rear waist region, an outer cover including a front waist region outer cover portion and rear waist region outer cover portion of a printed waistband, a liner generally parallel to the outer cover, and an absorbent core disposed therebetween, the chassis having a chassis first lateral edge, a chassis second lateral edge, and a chassis waist edge; and
a first side panel including a first side panel portion of the printed waistband , the first side panel having a first side panel waist edge and a first side panel medial edge, the first side panel disposed adjacent the chassis such that first side panel medial edge is affixed to the chassis lateral edge,
wherein the front waist region outer cover portion and the first side panel portion each have a distal edge and a proximal edge, and wherein either the distal edge of the outer cover portion and the distal edge of the first side panel portion have a misalignment offset of up to 100 percent of the average waistband height, or the proximal edge of the outer cover portion and the proximal edge of the first side panel portion have a misalignment offset of up to 100 percent of the average waistband height, where the average waistband height is the average of the longitudinal height of the front waist region outer cover portion and the longitudinal height of the rear waist region outer cover portion.

12. The article of claim 11, wherein either the distal edge of the outer cover portion and the distal edge of the first side panel portion have a misalignment offset of between 5 percent and 100 percent of the average waistband height, or the proximal edge of the outer cover portion and the proximal edge of the first side panel portion have a misalignment offset of between 5 percent and 100 percent of the average waistband height.

13. The article of claim 11, wherein either the distal edge of the outer cover portion and the distal edge of the first side panel portion have a misalignment offset of between 10 percent and 100 percent of the average waistband height, or the proximal edge of the outer cover portion and the proximal edge of the first side panel portion have a misalignment offset of between 10 percent and 100 percent of the average waistband height.

14. The article of claim 11, wherein the chassis further includes a chassis portion of a leg band, wherein the first side panel further includes a first side panel portion of a leg band.

15. The article of claim 11, further comprising:
a second side panel including a second side panel portion of the waistband, the second side panel having a second side panel waist edge and a second side panel medial edge, the second side panel disposed adjacent the chassis such that second side panel medial edge is affixed to the chassis second lateral edge,
wherein the second side panel portion has a distal edge and a proximal edge, and wherein either the distal edge of the outer cover portion and the distal edge of the second side panel portion have a misalignment offset of up to 100 percent of the average waistband height, or the proximal edge of the outer cover portion and the proximal edge of the second side panel portion have a misalignment offset of up to 100 percent of the average waistband height.

16. The article of claim 11, wherein the outer cover portion and the first side panel portion have no lateral gap therebetween.

17. The article of claim 11, wherein the longitudinal height of the front waist region outer cover portion is at least 5 percent of the total waistband height, where the total waistband height is the sum of the longitudinal height of the front waist region outer cover portion and the longitudinal height of the rear waist region outer cover portion.

18. The article of claim 17, wherein the longitudinal height of the front waist region outer cover portion is at least 10 percent of the total waistband height, where the total waistband height is the sum of the longitudinal height of the front waist region outer cover portion and the longitudinal height of the rear waist region outer cover portion.

19. The article of claim 17, wherein the longitudinal height of the front waist region outer cover portion is at least 25 percent of the total waistband height, where the total waistband height is the sum of the longitudinal height of the front waist region outer cover portion and the longitudinal height of the rear waist region outer cover portion.

20. A disposable absorbent article having multiple printed components, the article comprising:
a chassis having an outer cover including an outer cover portion of a first printed band feature, a liner generally parallel to the outer cover, and an absorbent core disposed therebetween, the chassis having a chassis first lateral edge, a chassis second lateral edge, and a chassis waist edge; and
a first side panel including a first side panel portion of a first printed band feature, the first side panel having a first side panel waist edge and a first side panel medial edge, the first side panel disposed adjacent the chassis such that first side panel medial edge is affixed to the chassis lateral edge,
wherein the outer cover portion and the first side panel portion each have a longitudinal height, and wherein the longitudinal height of the outer cover portion and the longitudinal height of the first side panel portion are different by at least 25 percent of the longitudinal height of the outer cover portion.

21. The article of claim 20, wherein the longitudinal height of the outer cover portion and the longitudinal height of the first side panel portion are different by at least 50 percent of the longitudinal height of the outer cover portion.

22. The article of claim 20, wherein the outer cover portion and the first side panel portion have no lateral gap therebetween.

23. A disposable absorbent article having multiple printed components, the article comprising:
a chassis having a front waist region, and rear waist region, an outer cover including a front waist region outer cover portion and rear waist region outer cover portion of a printed waistband, a liner generally parallel to the outer cover, and an absorbent core disposed therebetween, the chassis having a chassis first lateral edge, a chassis second lateral edge, and a chassis waist edge;
a first side panel including a first side panel portion of the printed waistband, the first side panel having a first side panel medial edge and a first side panel lateral edge, the first side panel disposed adjacent the chassis such that first side panel medial edge is affixed to the chassis lateral edge; and
a second side panel including a second side panel portion of the printed waistband, the second side panel having a second side panel medial edge and a second side panel lateral edge, the second side panel disposed adjacent the chassis such that second side panel medial edge is affixed to the chassis lateral edge and the second side panel lateral edge is coupled to the first side panel lateral edge,
wherein the first side panel portion and the second side panel portion each have a distal edge and a proximal edge, and wherein either the distal edge of the first side panel portion and the distal edge of the second side panel portion have a misalignment offset of up to 175 percent of the average waistband height, or the proximal edge of the first side panel portion and the proximal edge of the second side panel portion have a misalignment offset of up to 175 percent of the average waistband height, where the average waistband height is the average of the longitudinal height of the front waist region outer cover portion and the longitudinal height of the rear waist region outer cover portion.

24. The article of claim 23, wherein either the distal edge of the first side panel portion and the distal edge of the second side panel portion have a misalignment offset of up to 125 percent of the average waistband height, or the proximal edge of the first side panel portion and the proximal edge of the second side panel portion have a misalignment offset of up to 125 percent of the average waistband height.
